(19) United States
(12) Patent Application Publication
(10) Pub. No.: US 2025/0166180 A1
(43) Pub. Date: May 22, 2025

(54) INFORMATION PROCESSING METHOD, NON-TRANSITORY STORAGE MEDIUM, AND INFORMATION PROCESSING APPARATUS

(71) Applicants: Changhee HAN, Tokyo (JP); Tsz Tat CHU, Tokyo (JP)

(72) Inventors: Changhee HAN, Tokyo (JP); Tsz Tat CHU, Tokyo (JP)

(21) Appl. No.: 18/946,353

(22) Filed: Nov. 13, 2024

(30) Foreign Application Priority Data
Nov. 17, 2023 (JP) 2023-196090
Sep. 6, 2024 (JP) 2024-154344

(51) Int. Cl. G06T 7/00; G06V 10/764; G16H 10/60; G16H 30/20; G16H 30/40; G16H 50/20

(52) U.S. Cl. CPC G06T 7/0012; G06V 10/764; G16H 10/60; G16H 30/20; G16H 30/40; G16H 50/20; G06T 2207/30096

(57) ABSTRACT

An information processing method according to an aspect of the present disclosure causes a computer to execute operations to thereby generate data for medical AI/drug discovery AI, the operations including: acquiring multiple image information (acquisition unit), in which each of the multiple image information is generated by a medical device having one or more imaging functions and private information contained in such image information has been subjected to an anonymization process (anonymization unit); acquiring multiple clinical information related to medical practice for a patient, in which private information associated with the multiple clinical information has been anonymized; selecting target image information suitable as data for medical AI/drug discovery AI from among the multiple image information, selecting target clinical information to be used as the data for medical AI/drug discovery AI from among the multiple clinical information (selection unit); associating disease information related to a disease and organ information related to an organ with the target image information and the target clinical information (association unit); and performing standardization on the target image information and the target clinical information (standardization unit).

### BACKGROUND

### Field

The present disclosure relates to an information processing method, a non-transitory storage medium, and an information processing apparatus.

### Description of Related Art

A technique for processing data related to medical care is conventionally known. For example, JP2022-166318 A describes a system for processing medical information, in which the system detects regions where a healthcare institution with clinical data sets confidential items based on such region. However, the technique described in the above document cannot sufficiently improve the efficiency in which data is utilized in medical AI/drug discovery AI.

### SUMMARY

An object of the present disclosure is to improve the efficiency in which data is utilized in medical AI/drug discovery AI.

An information processing method according to an aspect of the present disclosure causes a computer to execute operations to thereby generate data for medical AI/drug discovery AI, the operations including: acquiring multiple image information, in which each of the multiple image information is generated by a medical device having one or more imaging functions and private information contained in such image information has been subjected to an anonymization process; acquiring multiple clinical information related to medical practice for a patient, in which private information associated with the multiple clinical information has been anonymized; selecting target image information suitable as data for medical AI/drug discovery AI from among the multiple image information; selecting target clinical information to be used as the data for medical AI/drug discovery AI from among the multiple clinical information; associating, as needed, disease information related to a disease and organ information related to an organ with the target image information and the target clinical information; and performing standardization on the target image information and the target clinical information.

A non-transitory storage medium according to another aspect of the present disclosure stores a program which causes a computer to execute operations to thereby generate data for medical AI/drug discovery AI, the operations including: acquiring multiple image information, wherein each of the multiple image information is generated by a medical device having one or more imaging functions and private information contained in such image information has been subjected to an anonymization process; acquiring multiple clinical information related to medical practice for a patient, wherein private information associated with the multiple clinical information has been anonymized; selecting target image information suitable as data for medical AI/drug discovery AI from among the multiple image information; selecting target clinical information to be used as the data for medical AI/drug discovery AI from among the multiple clinical information; associating, as needed, disease information related to a disease and organ information related to an organ with the target image information and the target clinical information; and performing standardization on the target image information and the target clinical information.

An information processing apparatus according to another aspect of the present disclosure includes a computer that executes operations to thereby generate data for medical AI/drug discovery AI, the operations including: acquiring multiple image information, wherein each of the multiple image information is generated by a medical device having one ore more imaging functions and private information contained in such image information has been subjected to an anonymization process; acquiring multiple clinical information related to medical practice for a patient, wherein private information associated with the multiple clinical information has been anonymized; selecting target image information suitable as data for medical AI/drug discovery AI from among the multiple image information; selecting target clinical information to be used as the data for medical AI/drug discovery AI from among the multiple clinical information; associating, as needed, disease information related to a disease and organ information related to an organ with the target image information and the target clinical information; and performing standardization on the target image information and the target clinical information.

### BRIEF DESCRIPTION OF DRAWINGS

FIG. 1 is a conceptual diagram of a system 1.

FIG. 2 is a diagram illustrating an example of a functional configuration of the system 1.

FIG. 3 is a diagram illustrating an example of an operation of an information processing apparatus 2.

FIG. 4 is a diagram illustrating an example of a hardware configuration of each apparatus in the system 1.

### DETAILED DESCRIPTION

### <1. Outline>

A system 1 according to the present embodiment (hereinafter simply referred to as the “system 1”) is an all-in-one platform for research and development in the field of medical or drug discovery image processing. The system 1 is capable of generating high-quality training/inference data for medical AI and drug discovery AI, by performing (1) data collection, (2) data selection, (3) annotation, if needed, and (4) standardization.

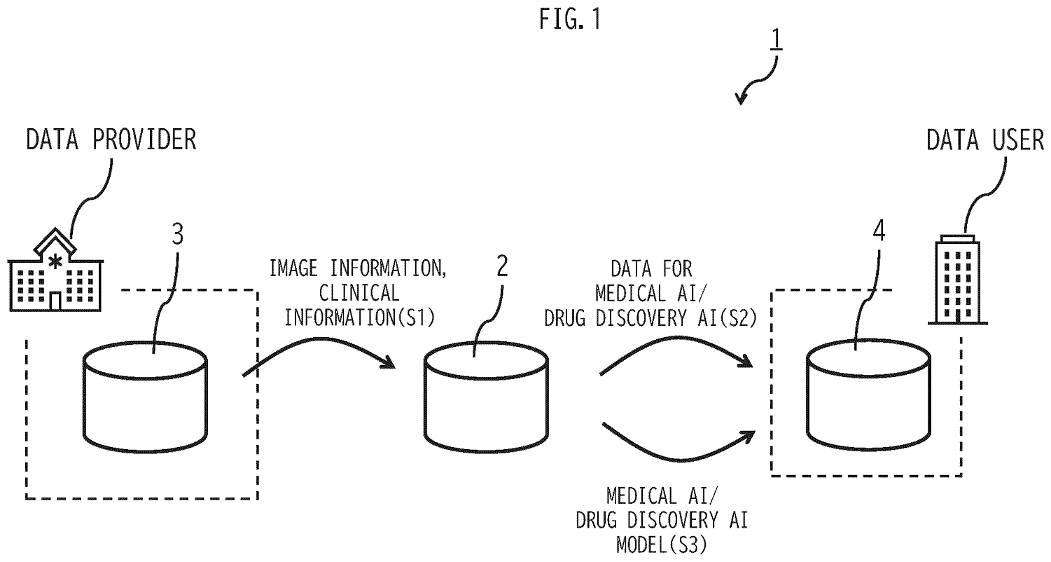

An example of an operation of the system 1 will be described below, referring to FIG. 1. The system 1 includes a data provider apparatus 3, an information processing apparatus 2, and a data user apparatus 4. In an embodiment, the data provider apparatus 3 is a server apparatus managed by a data provider. The data provider may be, a healthcare institution, such as a hospital. In an embodiment, the data user apparatus 4 may be, for example, a server apparatus managed by a data user. The data user may be, for example, an AI development company, a medical device development company, a pharmaceutical company, a research institution.

The information processing apparatus 2 acquires multiple image information and multiple clinical information from the data provider apparatus 3 (S1). The multiple image information are generated by a medical device having imaging functions (hereinafter referred to as the “medical imaging apparatus”). The data provider apparatus 3 may receive image information from the medical imaging apparatus, and transmit such image information to the information processing apparatus 2. The multiple clinical information may include, for example, information related to medical practice for patients at a healthcare institution.

In an embodiment, the medical imaging apparatus is an apparatus for photographing a body of a patient or a test subject (hereinafter referred to as the “subject”). The medical imaging apparatus may be, for example, a CT (Computed Tomography) scanner, an MRI (Magnetic Resonance Imaging) scanner, an X-ray scanner, an ultrasound scanner, an endoscope scanner, a PET (Position Emission Tomography) scanner, a WSI (Whole Slide Imaging) scanner, etc.

The information processing apparatus 2 generates data for medical AI/drug discovery AI by performing data selection, annotation and standardization with respect to the multiple image information and the multiple clinical information obtained from the data provider apparatus 3. The information processing apparatus 2 transmits such data for medical AI/drug discovery AI to the data user apparatus 4 (S2). The information processing apparatus 2 may also provide the data user apparatus 4 with an AI model which has been trained on the data for medical AI/drug discovery AI (S3).

The system 1 can enable the data user to easily access the data for medical AI/drug discovery AI. In the system 1, the information processing apparatus 2 may be regarded as a data hub.

Now, the configuration and operation of the system 1 will be described in more detail below.

### <2. Functional Configuration>

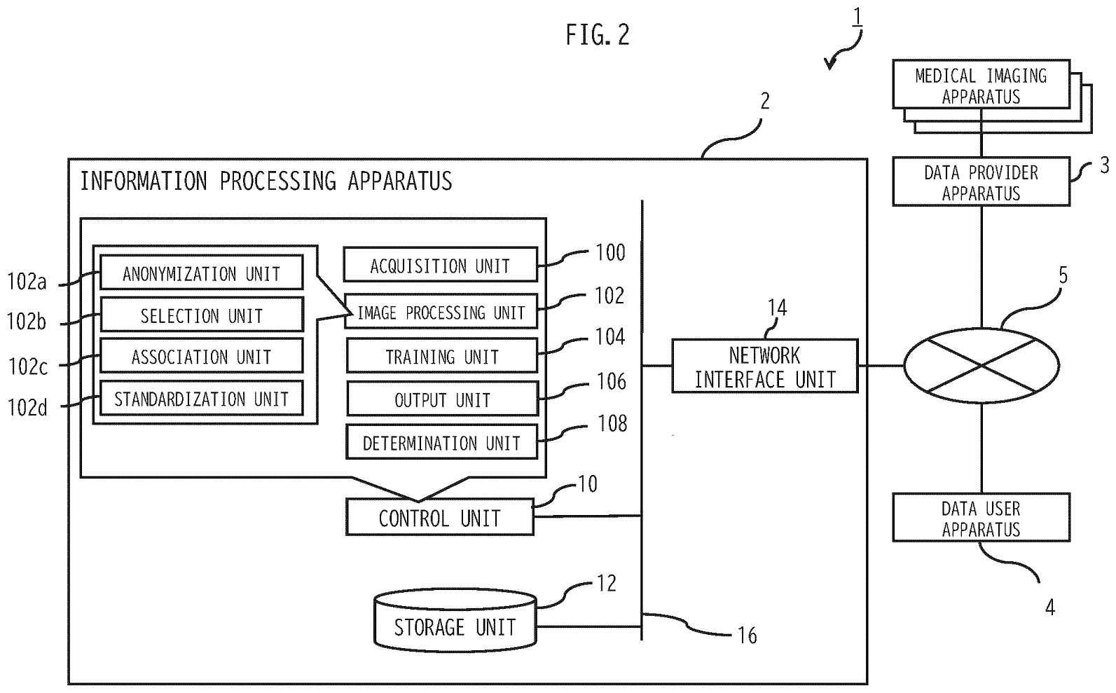

The functional configuration of the system 1 will be described below, referring to FIG. 2. In the system 1, the information processing apparatus 2 is configured so as to be communicable with the data provider apparatus 3 and the data user apparatus 4 via a communication network 6.

### [Information Processing Apparatus 2]

The information processing apparatus 2 is a server apparatus that generates data for medical AI/drug discovery AI. The information processing apparatus 2 includes a control unit 10, a storage unit 12, a network interface unit 14 and a bus 16. The control unit 10, the storage unit 12 and the network interface unit 14 are electrically connected to each other via the bus 16.

In the present disclosure, “an apparatus generating data or information” may refer to either of a feature in which the data or information that has resulted from pre-determined computation is made processable by a control unit of such apparatus or a feature in which the data or information that has resulted from pre-determined computation is stored in a storage unit of such apparatus.

### (Control Unit 10)

The control unit 10 functions as an acquisition unit 100, an image processing unit 102, a training unit 104, an output unit 10 and a determination unit 108, by executing various types of programs stored in the storage unit 12 (described later).

### -Acquisition Unit 100-

The acquisition unit 100 acquires multiple image information generated by the medical imaging apparatus. The image information may be image files in formats, such as DICOM (Digital Imaging and Communications in Medicine), NIfTI (Neuroimaging Informatics Technology Initiative), TIFF (Tagged Image File Format), SVS, NDPI.

The acquisition unit 100 may directly receive the multiple image information from the medical imaging apparatus or may alternatively receive the multiple image information that have been accumulated in a server apparatus that can communicate with the medical imaging apparatus.

The acquisition unit 100 further acquires multiple clinical information related to medical practice for a patient. In an embodiment, the acquisition unit 100 directly or indirectly acquires clinical information from a server managed by a healthcare institution, such as a hospital. The clinical information may include, for example, information related to age, sex, height, weight, medical history/family history, surgical history, major complaint, major symptoms/progress, nursing observation record, referring department, diagnosis, findings, molecular diagnostic results. The clinical information may be, for example, a file containing text, such as an Excel (registered trademark) file or a CSV file.

In the present disclosure, the “acquisition of information” includes making such information processable in the control unit 10. The “acquisition of information” may be any of receiving such information from another apparatus, reading such information form the storage unit 12, and obtaining such information as a result of pre-determined processing.

### -Image Processing Unit 102-

The image processing unit 102 generates data for medical AI/drug discovery AI from the multiple image information. The image processing unit 102 includes an anonymization unit 102a, a selection unit 102b, an association unit 102c, and a standardization unit 102d.

### -Anonymization Unit 102a-

The anonymization unit 102a applies an anonymization process to private information that is contained in each of the multiple image information acquired by the acquisition unit 100. The anonymization unit 102a also anonymizes the private information associated with the multiple clinical information acquired by the acquisition unit 100. Applying the anonymization process to the private information contained in the image information and anonymizing the private information associated with the clinical information includes rendering such private information incomprehensible or difficult-to-comprehend by a human or a computer. The anonymization unit 102a applying the anonymization process to the private information contained in the image information may include, for example, deleting or applying blurring to face information in an image that contains a face of a human who was a photography subject or replacing such face information with face information of another person's face. The anonymization unit 102a anonymizing the private information associated with the clinical information may include, for example, deleting such private information, encrypting such private information, substituting alternative texts for such private information.

### -Selection Unit 102b-

The selection unit 102b selects target image information suitable as the data for medical AI/drug discovery AI from among the multiple image information acquired by the acquisition unit 100.

In an embodiment, the selection unit 102b selecting the target image information from among the multiple image information is performed based on an operation on a terminal apparatus used by a user (e.g., a doctor). In an embodiment, such terminal apparatus displays the multiple image information and receives, from the user, an operation related to selection of image information that is suitable as the data for medical AI/drug discovery AI. The terminal apparatus then transmits information related to the selected image information to the information processing apparatus 2. The selection unit 102b of the information processing apparatus 2 receives such information and selects, as the target image information, the image information that has been selected by the user from among the multiple image information.

In an embodiment, the selection unit 102b selecting the target image information from the multiple image information includes determining whether or not contained lesions have a complication. The selection unit 102b may determine whether or not the contained lesions have a complication based on, for example, an input from a user who has visually observed the image information. More specifically, the selection unit 102b may select, as the target image information, image information which has been determined by the user as having no lesion having a complication, from among the multiple image information. In addition, the selection unit 102b may select, as the target image information, image information in which a single disease has been specified by a pre-determined classification model, from among the multiple image information. The classification model may be based on, for example, SVM (Support Vector Machine), deep learning, and decision tree.

The selection unit 102b selects target clinical information to be used as the data for medical AI/drug discovery AI, from among the multiple clinical information acquired by the acquisition unit 100. In one example, in a case where the clinical information contains multiple items and values corresponding to the respective items, the selection unit 102b may select, as the target clinical information, clinical information in which a value corresponding to a pre-determined item has been set, from among the multiple clinical information. Here, when the clinical information contains information such as, for example, “age: 30” and “diagnosis: lung cancer,” the “age” and the “diagnosis” fall under the items and the “30” and the “lung cancer” fall under the values corresponding to the respective items. For example, when the pre-determined item includes an “age” and a “diagnosis,” the selection unit 102b selects, as the target clinical information, the clinical information in which values corresponding to the “age” and the “diagnosis,” respectively, have been set (i.e., such values are not blank, unfilled, default values, or the like), from among the multiple clinical information. The pre-determined item may be, for example, age, sex, height, weight, medical history/family history, surgical history, major complaint, major symptoms/progress, nursing observation record, referring department, diagnosis, findings, molecular diagnostic results.

### -Association Unit 102c-

The association unit 102c associates disease information related to a disease and organ information related to an organ with the target image information and the target clinical information. The association unit 102c associating the disease information with the target image information and the clinical information may individually associate the disease information with each of the target image information and the target clinical information or may associate the disease information with a set of target image information and target clinical information.

The association unit 102c may associate the disease information and the organ information with the target image information by, for example, analyzing a specific pattern or characteristic contained in the target image information. Such analysis may be performed using a pre-determined classification model. The association unit 102c may associate the disease information and the organ information with the target image information based on an input from the user who has visually observed the target image information. The association of the disease information and the organ information with the target image information may be referred to as “annotation”.

In an embodiment, the association unit 102c associating the disease information and the organ information with the target image information includes specifying the locations of lesions or organ structures contained in the target image information. Specifying the locations of lesions or organ structures contained in the target image information includes, in the target image information, drawing contours of organ structures which contain lesions or a disease, coloring portions in the organ structures where lesions or a disease exist, coloring portions in each organ structure irrespective of lesions or a disease. Specifying the locations of lesions or organ structures contained in the target image information includes filling such locations (or regions including such locations) with a color in the target image information. Specifying the locations of lesions or organ structures contained in the target image information may be referred to as “segmentation”.

In an embodiment of the present invention, the association unit 102c associating the disease information and the organ information with the target image information includes specifying the classification of lesions or organ structures contained in the target image information. Specifying the classification of a lesion may include, for example, specifying the name of the disease corresponding to such lesions, specifying the degree of progression of such lesions (e.g., a cancer stage).

The association unit 102c may associate, based on at least part of the target clinical information, the disease information with such target clinical information. For example, if the target clinical information contains findings, the association unit 102c may specify the disease information based on such findings and associate the specified disease information with the target clinical information. If the target clinical information contains the name of a diagnosis, the association unit 102c may associate the name of the diagnosis, as the disease information, with the target clinical information.

In an embodiment, at least either the association of the disease information and the organ information with the target image information or the target clinical information may be double-checked by an expert physician (e.g., a radiologist or a physician). More specifically, the system 1 may be configured such that a first user (i.e., a healthcare professional) associates disease information with target image information and target clinical information, and a second user (i.e., a expert physician), having greater expert knowledge than the first user, then checks the validity of such association. In such case, the information processing apparatus 2 may be configured so as to generate data for medical AI/drug discovery AI based on the target image information and the target clinical information whose association have been determined as valid by the second user.

### -Standardization Unit 102d-

The standardization unit 102d performs standardization on the target image information and the target clinical information. The standardization of the target image information may include at least any of noise removal, contrast adjustment, edge enhancement, and color compensation. The standardization of the target clinical information may include extracting part of the data contained in the clinical information (e.g., extracting, from the clinical information, age, sex, height, weight, medical history/family history, surgical history, major complaint, major symptoms/progress, nursing observation record, referring department, diagnosis, findings, molecular diagnostic results), unifying the format or unit thereof. The standardization of the target image information and the target clinical information (in particular, the standardization for the target clinical information) by the standardization unit 102d is particularly important in order to achieve the objective of collecting information from multiple healthcare institutions and promoting the utilization of such information. The standardization may also be referred to as “pre-processing” or “normalization”.

The standardization unit 102d performing standardization on the target image information includes aligning multiple target image information generated as a result of the photographing of a common subject by mutually different medical imaging apparatuses (which can also be referred to as “paired scanning”). More specifically, in a situation where the selection unit 102b selects, from among the multiple image information, the first target image information generated by the first medical device and the second target image information generated by the second medical device, the standardization unit 102d performing standardization on the target image information includes aligning the first target image information and the second target image information. For example, in a situation where the selection unit 102b selects MR scans (corresponding to the first target image information) generated as a result of the photographing of a certain subject by an MRI scanner (corresponding to the first medical device) and CT scans (corresponding to the second target image information) generated as a result of the photographing of the same subject by a CT scanner (corresponding to the second medical device), the standardization unit 102d performing standardization on the target image information includes aligning the MRI scans and the CT scans.

In the present disclosure, “aligning the first target image information and the second target image information” may refer to any of associating the first target image information and the second target image information with each other, combining the first target image information and the second target image information with each other, and comparing the target image information and the second target image information.

The standardization unit 102d performing standardization on the target image information includes aligning multiple target image information generated as a result of the photographing of a common subject using a common medical imaging apparatus at different points in time. More specifically, in a situation where the selection unit 102b selects, from among the multiple image information, the first target image information generated based on photographing, by a pre-determined medical device at a first point in time, and the second target image information generated based on photographing, by the pre-determined medical device at a second point in time different from the first point in time, the standardization unit 102d performing standardization on the target image information includes aligning the first target image information and the second target image information.

In this way, by aligning multiple target image information, it is possible to generate more useful data for medical AI/drug discovery AI. More specifically, multiple target image information generated as a result of the photographing of a common subject by using multiple mutually different medical imaging apparatuses may have properties that are complementary to each other.

Regarding the alignment of multiple target image information that have been generated as a result of the photographing of a common subject by using a common medical imaging apparatus at different points in time, the multiple target image information may indicate a change along a time series. Therefore, the alignment of multiple target image information enables the generation of more useful data for medical AI/drug discovery AI, in the same way as in the above case.

The image processing unit 102 applies the anonymization unit 102a, the selection unit 102b, the association unit 102c and the standardization unit 102d to the multiple image information to thereby generate data for medical AI/drug discovery AI. More specifically, the data for medical AI/drug discovery AI contains information which has been obtained by associating the disease information and the organ information with the target image information selected from among the multiple image information whose private information has been anonymized and by further applying standardization thereto.

### -Training Unit 104-

The training unit 104 causes the data generated by the image processing unit 102 to be trained by a medical AI/drug discovery AI model. The training model may be any of neural network (including transformer, generative adversarial network, convolutional neural network), SVM, and decision tree.

### -Output Unit 106-

The output unit 106 outputs at least one of the data for medical AI/drug discovery AI obtained by the image processing unit 102 and the medical AI/drug discovery AI model obtained by the training unit 104.

In the present disclosure, “outputting information” may be any of displaying such information, outputting it as sounds, and transmitting it to another apparatus. In the present disclosure, a certain apparatus transmitting information to another apparatus is not limited to the certain apparatus transmitting such information to the other apparatus by way of direct communication, but also includes the certain apparatus transmitting such information to the other apparatus via a communication network 6 and transmitting such information to the other apparatus via an apparatus that relays information.

### -Determination Unit 108-

The determination unit 108 determines a data provision fee to be paid to a data provider. More specifically, when the output unit 106 transmits the data for medical AI/drug discovery AI or the medical AI/drug discovery AI model to a user, the determination unit 108 determines at least part of a usage fee for the data for medical AI/drug discovery AI or the medical AI/drug discovery AI model which has been received (or to be received) from the user of such data, as a data provision fee to be paid to the data provider of the target image information/clinical information.

The determination unit 108 may take into account multiple factors when determining the data provision fee. For example, the determination unit 108 may calculate the data provision fee based on, for example: the quantity and quality of the target image information provided by the data provider; the rarity of the target image information provided by the data provider (e.g., the rarity of a disease or drug contained in such data); the presence or absence of clinical information or annotation associated with the target image information, as well as the type and quality thereof; the number of times that the data for medical AI/drug discovery AI based on the target image information/clinical information provided by the data provider has been used; the functionality of the medical AI/drug discovery AI model based on the data for the medical AI/drug discovery AI; and the scale and/or commerciality of the data provider (e.g., whether the data provider is a large-sized enterprise, a small or middle-sized enterprise, or a research institution). These factors may be adjusted as appropriate in order to ensure the fairness and appropriateness of the data provision fee.

### (Storage Unit 12)

The storage unit 12 stores various types of information that should be stored for the information processing apparatus 2 to operate. The storage unit 12 stores various types of programs that are executed by the control unit 10.

### (Network Interface Unit 14)

The network interface unit 14 realizes communication with another apparatus or a system via the communication network 6.

### [Data Provider Apparatus 3]

In an embodiment, the data provider apparatus 3 is a server apparatus that is managed by a data provider. The data provider apparatus 3 is configured so as to be communicable with multiple medical imaging apparatuses. The data provider apparatus 3 can transmit image information received from multiple medical imaging apparatuses to the information processing apparatus 2 via the communication network 6.

### [Data User Apparatus 4]

In one embodiment, the data user apparatus 4 is a server apparatus that is managed by a data user. The data user apparatus 4 can receive at least one of the data for medical AI/drug discovery AI and the medical AI/drug discovery AI model from the information processing apparatus 2 via the communication network 6.

### [Communication Network 6]

The communication network 6 realizes communication among the information processing apparatus 2, the data provider apparatus 3 and the data user apparatus 4. The communication network 6 may achieve communication based on, for example, a TCP/IP protocol. Although not shown in FIG. 2, the system 1 may include multiple data provider apparatuses 3 and multiple data user apparatuses 4. In such case, the information processing apparatus 2 may acquire multiple image information from each of multiple data provider apparatuses 3.

### <3. Operation>

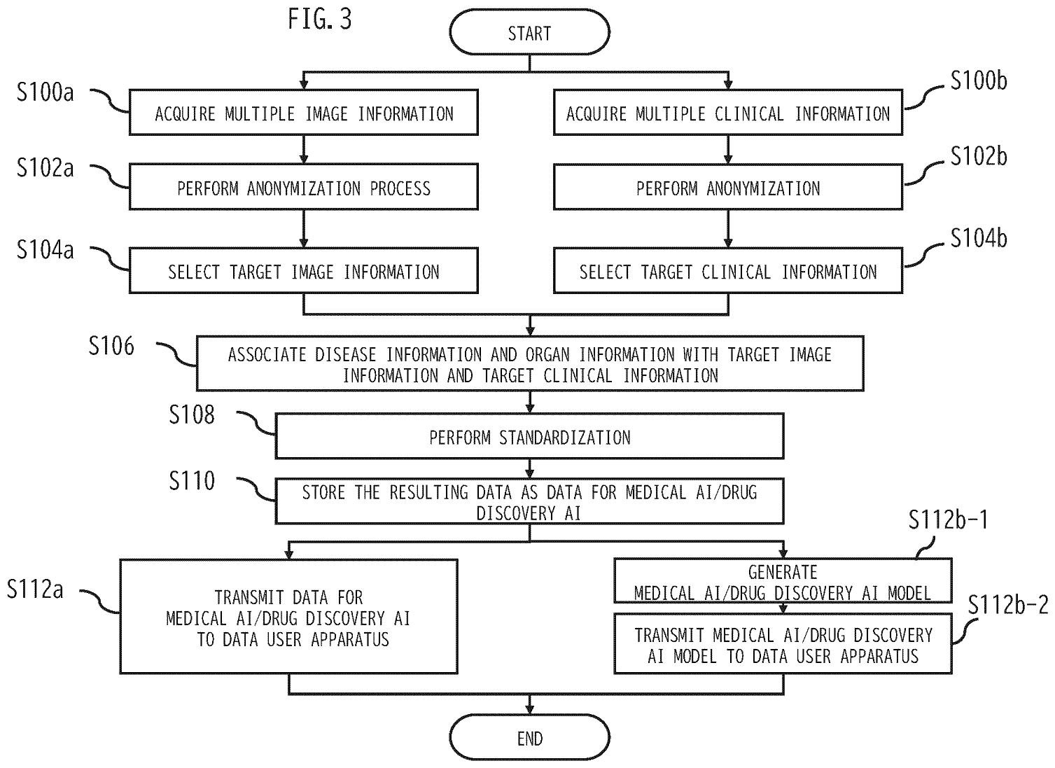

An example of the operation of the information processing apparatus 2 will now be described below, referring to FIG. 3.

First, the information processing apparatus 2 acquires multiple image information from the data provider apparatus 3 (S100a). Next, the information processing apparatus 2 applies an anonymization process to each of the multiple image information (S102a). The information processing apparatus 2 then selects target image information suitable as data for medical AI/drug discovery AI from among the multiple image information (S104a). Similarly to the above, the information processing apparatus 2 acquires multiple clinical information from the data provider apparatus 3 (S100b), performs anonymization (S102b), and selects target clinical information (S104b). Next, the information processing apparatus 2 associates disease information and organ information with the target image information and the target clinical information (S106). The information processing apparatus 2 then applies standardization to the target image information (S108). The information processing apparatus 2 then stores, in the storage unit 12, the data for medical AI/drug discovery AI generated as a result of the process of S108 (S110). The information processing apparatus 2 may transmit the stored data for medical AI/drug discovery AI to the data user apparatus 4 (S112a) or use such data in order to generate a medical AI/drug discovery AI model (S112b-1). If the medical AI/drug discovery AI model is generated, such medical AI/drug discovery AI model is transmitted to the data user apparatus 4 (S112b-2).

### <4. Hardware Configuration>

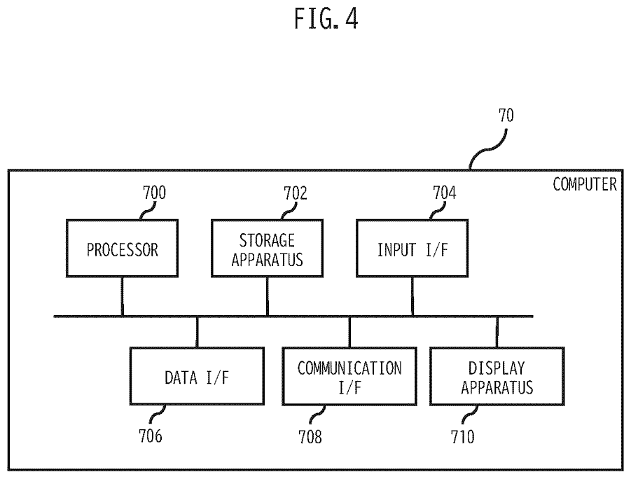

The following description will describe, referring to FIG. 4, an example of a hardware configuration in the case of implementing the above-described apparatuses included in the system 1 with a computer 70. It should be noted that the function of each of the apparatuses may be implemented so as to be distributed to multiple apparatuses.

As shown in FIG. 4, the computer 70 includes a processor 700, a storage apparatus 702, an input I/F 704, a data I/F 706, a communication I/F 708 and a display apparatus 710.

The processor 700 executes programs stored in the storage apparatus 702 to thereby control various types of processing in the computer 70. For example, each of the functional units included in the control unit 10 of the information processing apparatus 2 may be implemented by executing, by the processor 700, the relevant program stored in the storage apparatus 702.

The storage apparatus 702 may be a storage medium such as, for example RAM (Random Access Memory). The RAM temporarily stores a program code of a program to be executed by the processor 700 and data that is necessary for executing the program.

The storage apparatus 702 may also be a nonvolatile storage medium such as, for example, a hard disk drive (HDD) and a flash memory. The storage apparatus 702 stores an operating system and various programs for implementing each of the above-described configurations. The storage medium storing such various programs may be a non-transitory computer readable medium. In addition, the storage apparatus 702 may also store tables having various types of information registered therein and a database for managing such tables. Such programs and data are loaded to the storage apparatus 702 as needed, whereby such programs and data are referred to by the processor 700.

The input I/F 704 is a device for receiving input from a user. Specific examples of the input I/F 704 may include a camera, a button, a microphone, a keyboard, a mouse, a touch screen, various sensors, a wearable device. The input I/F 704 is connected to the computer 70 via an interface, such as a USB (Universal Serial Bus).

The data I/F 706 is a device for allowing data to be input from the outside of the computer 70. Specific examples of the data I/F 706 may include a drive apparatus. for reading data stored in various types of storage media. The data I/F 706 may be provided outside the computer 70. In such case, the data I/F 706 is connected to the computer 70 via an interface, such as a USB.

The communication I/F 708 is a device for allowing wired or wireless data communication with an external apparatus of the computer 70 via the communication network 6. The communication I/F 708 may be provided outside the computer 70. In such case, the data I/F 708 is connected to the computer 70 via an interface, such as a USB.

The display apparatus 710 is a device for displaying various types of information. Specific examples of the display apparatus 710 may include a liquid crystal display, an organic EL (Electro-Luminescence) display, a display of a wearable device. The display apparatus 710 may be provided outside the computer 70. In such case, the display apparatus 710 is connected to the computer 70 via, for example, a display cable. When a touch screen is employed as the input I/F 704, the display apparatus 710 may be configured in an integral manner with the input I/F 704.

Regarding the components provided in each of the apparatuses included in the system 1 described in the above-described embodiments, when a program stored in the storage apparatus 702 is executed by the processor 700, specific processing is performed in cooperation with other hardware. In other words, such components may each be assumed to be software, firmware, or hardware corresponding thereto and, under the concept of both the software/firmware and the hardware, such components may each be referred to as, or replaced with, a “function,” “means,” a “part,” a “processing circuitry,” a “unit,” or a “module.”

### <5. Modification>

The features described in the embodiments above may be modified as appropriate to the extent that no contradiction is introduced.

The information processing apparatus 2 may further be provided with a function of recording information related to the transmission and receipt of the data for medical AI/drug discovery AI in Blockchain. The information related to the transmission and receipt of the data for medical AI/drug discovery AI may be the data for the medical AI/drug discovery AI itself, or a history of transmission and receipt of the data for medical AI/drug discovery AI, or a NFT (Non-Fungible Token) for accessing the data for medical AI/drug discovery AI. The information processing apparatus 2 may, for example, store the data for medical AI/drug discovery AI in Blockchain, so that the data user apparatus 4 can access the data for medical AI/drug discovery AI on such Blockchain. Alternatively, for example, the image processing apparatus 2 may transfer a NFT to the data user apparatus 4 on Blockchain, so that the data user apparatus can access the data for medical AI/drug discovery AI corresponding to such NFT. Such configuration makes it possible to enhance security related to the use of the data for medical AI/drug discovery AI. In another embodiment, the information processing apparatus 2 may record the data for medical AI/drug discovery AI, or the information related to the transmission and receipt thereof, in an IPFS (InterPlanetary File System).

### <6. Aspects of Implementations of the Present Disclosure>

The embodiments described above are merely examples of the present disclosure and they in no way limit the scope of application of the present disclosure. The apparatuses included in the system 1 may be physically-existing apparatuses or so-called virtual machines. The apparatuses included in the system 1 may be constituted by multiple apparatuses that are physically or logically separated. For example, part of the functions of the information processing apparatus 2 may be implemented in the first information processing apparatus and the other part thereof may be implemented in the second information processing apparatus that is operable in cooperation with the first information processing apparatus. In addition, the apparatuses included in the system 1 may be apparatuses on a cloud (i.e., a computing resource that is available via a network).

The present disclosure may encompass, for example, the aspects of implementations set forth below. The items in parentheses indicate corresponding configurations in the above-described embodiments.

### [Appendix 1]

An information processing method according to an aspect of the present disclosure causes a computer 70 to execute operations to thereby generate data for medical AI/drug discovery AI, the operations including: acquiring multiple image information (acquisition unit 100), in which each of the multiple image information is generated by a medical device having one or more imaging functions and private information contained in such image information has been anonymized; acquiring multiple clinical information related to medical practice for a patient, in which private information associated with the multiple clinical information has been anonymized (anonymization unit 102a); selecting target image information suitable as data for medical AI/drug discovery AI from among the multiple image information and selecting target clinical information to be used as the data for medical AI/drug discovery AI from among the multiple clinical information (selection unit 102b); associating disease information related to a disease and organ information related to an organ with the target image information and the target clinical information (association unit 102c); and performing standardization on the target image information and the target clinical information (standardization unit 102d).

### [Appendix 2]

In the information processing method according to appendix 1, associating the disease information related to a disease and the organ information related to an organ with the target image information (association unit 102c) may include specifying one or more locations of and/or classifying one or more lesions and/or one or more organ structures contained in the target image information.

### [Appendix 3]

In the information processing method according to appendix 1 or 2, selecting the target image information from among the multiple image information (selection unit 102b) may be performed based on an operation on a terminal apparatus that is used by a user.

### [Appendix 4]

In the information processing method according to any one of appendices 1 to 3, selecting the target image information from among the multiple image information (selection unit 102b) may include determining whether or not one or more contained lesions in each of the multiple image information have a complication.

### [Appendix 5]

In the information processing method according to any one of appendices 1 to 4, selecting the target image information from among the multiple image information (selection unit 102b) may include selecting, from among the multiple image information, first target image information generated by first medical device and second target image information generated by second medical device (selection unit 102b), and performing standardization on the target image information (standardization unit 102d) may include aligning the first target image information and the second target image information.

### [Appendix 6]

In the information processing method according to any one of appendices 1 to 5, selecting the target image information from among the multiple image information (selection unit 102b) may include selecting, from among the multiple image information, first target image information generated based on photographing by a pre-determined medical device at a first point in time and second target image information generated based on photographing by the pre-determined medical device at a second point in time different from the first point in time (selection unit 102b), and performing standardization on the target image information (standardization unit 102d) may include aligning the first target image information and the second target image information.

### [Appendix 7]

The information processing method according to any one of appendices 1 to 6 may further cause the computer 70 to execute recording information related to the transmission and receipt of the data for medical AI/drug discovery AI in Blockchain.

### [Appendix 8]

The information processing method according to any one of appendices 1 to 7 may further cause the computer 70 to execute generating a training model which has been trained on the data for medical AI/drug discovery AI (training unit 104).

### [Appendix 9]

The information processing method according to any one of appendices 1 to 8 may cause the computer 70 to execute: transmitting the data for medical AI/drug discovery AI or a medical AI/drug discovery AI model to a user (output unit 106); and determining at least part of a usage fee for the data for medical AI/drug discovery AI or the medical AI/drug discovery AI model which has been received from the user, as a data provision fee to be paid to a provider of the target image information (determination unit 108).

### [Appendix 10]

A non-transitory storage medium according to another aspect of the present disclosure stores a program which causes a computer 70 to execute operations to thereby generate data for medical AI/drug discovery AI, the operations including: acquiring multiple image information (acquisition unit 100), in which each of the multiple image information is generated by a medical device having one or more imaging functions and private information contained in such image information has been subjected to an anonymization process; acquiring multiple clinical information related to medical practice for a patient, in which private information associated with the multiple clinical information has been anonymized (anonymization unit 102a); selecting target image information suitable as data for medical AI/drug discovery AI from among the multiple image information and selecting target clinical information to be used as the data for medical AI/drug discovery AI from among the multiple clinical information (selection unit 102b); associating disease information related to a disease and organ information related to an organ with the target image information and the target clinical information (association unit 102c); and performing standardization on the target image information and the target clinical information (standardization unit 102d).

### [Appendix 11]

An information processing apparatus 2 according to another aspect of the present disclosure includes a computer 70 that executes operations to thereby generate data for medical AI/drug discovery AI, the operations including: acquiring multiple image information (acquisition unit 100), in which each of the multiple image information is generated by a medical device having one or more imaging functions and private information contained in such image information has been subjected to an anonymization process (anonymization unit 102a); acquiring multiple clinical information related to medical practice for a patient, in which private information associated with the multiple clinical information has been anonymized; selecting target image information suitable as data for medical AI/drug discovery AI from among the multiple image information and selecting target clinical information to be used as the data for medical AI/drug discovery AI from among the multiple clinical information (selection unit 102b); associating disease information related to a disease and organ information related to an organ with the target image information and the target clinical information (association unit 102c); and performing standardization on the target image information and the target clinical information (standardization unit 102d).

According to the present disclosure, it is possible to improve the efficiency in which data is utilized in medical AI/drug discovery AI.

What is claimed is:

1. An information processing method causing a computer to execute operations to thereby generate data for medical AI/drug discovery AI, the operations comprising:
acquiring multiple image information, in which each of the multiple image information is generated by a medical device having one or more imaging functions and private information contained in such image information has been subjected to an anonymization process;
acquiring multiple clinical information related to medical practice for a patient, in which private information associated with the multiple clinical information has been anonymized;
selecting target image information suitable as data for medical AI/drug discovery AI from among the multiple image information;
selecting target clinical information to be used as the data for medical AI/drug discovery AI from among the multiple clinical information;
associating disease information related to a disease and organ information related to an organ with the target image information and the target clinical information; and
performing standardization on the target image information and the target clinical information.

2. The information processing method according to claim 1, wherein associating the disease information related to a disease and the organ information related to an organ with the target image information includes specifying one or more locations of and/or classifying one or more lesions and/or one or more organ structures contained in the target image information.

3. The information processing method according to claim 1, wherein selecting the target image information from among the multiple image information is performed based on an operation on a terminal apparatus that is used by a user.

4. The information processing method according to claim 1, wherein selecting the target image information from among the multiple image information includes determining whether or not one or more contained lesions in each of the multiple image information have a complication.

5. The information processing method according to claim 1, wherein:
selecting the target image information from among the multiple image information includes selecting, from among the multiple image information, first target image information generated by a first medical device and second target image information generated by a second medical device, and
performing standardization on the target image information includes aligning the first target image information and the second target image information.

6. The information processing method according to claim 1, wherein:
selecting the target image information from among the multiple image information includes selecting, from among the multiple image information, first target image information generated based on photographing by a pre-determined medical device at a first point in time and second target image information generated based on photographing by the pre-determined medical device at a second point in time different from the first point in time; and
performing standardization on the target image information includes aligning the first target image information and the second target image information.

7. The information processing method according to claim 1, further causing the computer to execute recording information related to transmission and receipt of the data for medical AI/drug discovery AI in Blockchain.

8. The information processing method according to claim 1, further causing the computer to execute generating a training model which has been trained on the data for medical AI/drug discovery AI.

9. The information processing method according to claim 1, causing the computer to execute:
transmitting the data for medical AI/drug discovery AI to a user; and
determining at least part of a usage fee for the data for medical AI/drug discovery AI which has been received from the user, as a data provision fee to be paid to a provider of the target image information.

10. A non-transitory storage medium storing a program which causes a computer to execute operations to thereby generate data for medical AI/drug discovery AI, the operations comprising:
acquiring multiple image information, wherein each of the multiple image information is generated by a medical device having one or more imaging functions and private information contained in such image information has been subjected to an anonymization process;
acquiring multiple clinical information related to medical practice for a patient, wherein private information associated with the multiple clinical information has been anonymized;
selecting target image information suitable as data for medical AI/drug discovery AI from among the multiple image information;
selecting target clinical information to be used as the data for medical AI/drug discovery AI from among the multiple clinical information;
associating disease information related to a disease and organ information related to an organ with the target image information and the target clinical information; and
performing standardization on the target image information and the target clinical information.

11. An information processing apparatus, comprising a computer that executes operations to thereby generate data for medical AI/drug discovery AI, the operations including:
acquiring multiple image information, wherein each of the multiple image information is generated by a medical device having one or more imaging functions and private information contained in such image information has been subjected to an anonymization process;
acquiring multiple clinical information related to medical practice for a patient, wherein private information associated with the multiple clinical information has been anonymized;
selecting target image information suitable as data for medical AI/drug discovery AI from among the multiple image information;
selecting target clinical information to be used as the data for medical AI/drug discovery AI from among the multiple clinical information;
associating disease information related to a disease and organ information related to an organ with the target image information and the target clinical information; and
performing standardization on the target image information and the target clinical information.
